(19) United States
(12) Patent Application Publication
(10) Pub. No.: US 2026/0242711 A1
(43) Pub. Date: Aug. 20, 2026

(54) DEVICE AND METHOD FOR THE FLUID CONNECTION OF MICROFLUIDIC CONTAINERS

(71) Applicant: NETRI, Lyon (FR)

(72) Inventors: Serge Cyril ROUX, Lyon (FR); Thibault Frédéric Johan HONEGGER, Caluire (FR); Florian Arnaud Paul LARRAMENDY, Dardilly (FR)

(73) Assignee: NETRI, Lyon (FR)

(21) Appl. No.: 19/147,876

(22) PCT Filed: Jan. 16, 2024
(86) PCT No.: PCT/FR2024/050057

§ 371 Date: Jul. 14, 2025

(30) Foreign Application Priority Data
Jan. 16, 2023 (FR) FR2300385

(51) Int. Cl. C12M 1/00; C12M 3/00; C12M 3/06

(52) U.S. Cl. CPC C12M 23/40; C12M 21/08; C12M 23/16; C12M 29/00

(57) ABSTRACT

A unidirectional fluid connection device, the device including an intermediate fluid storage capacity which is configured to temporarily store a given volume of a fluid, the device including an inlet channel for the fluid coming into the device and an outlet channel for the fluid going out of the device, the device further including a first one-way valve which is configured to provide one-way fluid communication from the inlet channel to the storage capacity, the device further including a second one-way valve which is configured to provide one-way fluid communication from the storage capacity to the outlet channel.

The present invention concerns the field of microfluidic systems, and in particular of devices for connecting microfluidic systems.

Current multi-organ microfluidic systems, i.e., those enabling the culture of different organs, have limitations:
Multi-organ microfluidic systems, also known as multi-organ chips, are usually produced in complex dedicated chips, which do not allow studying one part of the system in isolation before studying the system as a whole. Any change to one part requires the design of a new custom chip.
Perfusion systems for circulating the culture medium between different organs involve large quantities of external tubes and pumps, making the setup complex and difficult to implement. In addition, these systems involve large dead volumes in the pipes, which waste molecules that are expensive to produce at the pre-clinical stage.
Multi-organ chips are currently not very compatible with standard microfluidic system formats, making high-throughput screening impossible, despite the growing market demand for systems generating large amounts of standardized and repeatable data.

In addition, in order to cultivate relevant endothelial barriers in microfluidic systems—which are also necessary to interface the multi-organ chips with a vascular network—a certain level of directional shear stress must be applied to the cells, to mimic the friction of blood—or blood cells—on endothelial cells, via their culture medium. This shear stress will give the endothelial cells their elongated appearance and confer the required porosity on the barrier. Providing shear stress is therefore a key element in enabling the formation of blood-brain barriers (BBBs), for example.

The disadvantages of current systems allowing generating shear stress are as follows:
Perfusion systems for circulating the culture medium—and generating shear stress in the process—are the same as the multi-organ microfluidic systems mentioned above and therefore have the same limitations;
Oscillating systems that tilt multi-organ microfluidic systems to generate gravity-induced flow eliminate the complexities of the perfusion systems but are limited by the low pressure (and therefore the low shear stress) that gravity can provide under these conditions, and deviate from in vivo conditions by applying shear stress in both directions instead of just one.

The invention therefore aims to propose a solution to all or part of these problems.

To this end, the present invention concerns a unidirectional fluid connection device, the device comprising an intermediate fluid storage capacity which is configured to temporarily store a given volume of a fluid, the device comprising an inlet channel for the fluid coming into the device and an outlet channel for the fluid going out of the device, the device further comprising a first one-way valve which is configured to provide one-way fluid communication from the inlet channel to the storage capacity, the device further comprising a second one-way valve which is configured to provide one-way fluid communication from the storage capacity to the outlet channel.

According to one embodiment, the invention comprises one or several of the following features, alone or in a technically acceptable combination.

According to one embodiment, the device is configured to form a unidirectional fluid connection from a first container to a second container, when the inlet channel is placed in fluid communication with the first container and when the outlet channel is placed in fluid communication with the second container.

According to these arrangements, the device is configured to transfer the given volume of the fluid from the first container to the second container, when the fluid connection from the first container to the second container is established with the device, and when the device is actuated by an actuator fluidically coupled to the intermediate fluid storage capacity, the actuator being configured to implement at least one suction and discharge cycle, the suction and discharge cycle comprising a generation of a suction vacuum towards the storage capacity of the device to suck the given volume of fluid from the first container to the intermediate storage capacity, followed by a generation of a discharge pressure in the storage capacity of the device to discharge the given volume of fluid from the intermediate storage capacity to the second container.

According to one embodiment, the first container is a first microfluidic reservoir, and the second container is a second microfluidic reservoir.

A reservoir is said to be microfluidic if it belongs to a microfluidic chip, or if it has the dimensions of a reservoir of a microfluidic chip. A microfluidic chip is composed of at least two reservoirs connected by at least one channel.

According to one embodiment, the given volume of transferred fluid is comprised between 5 μl and 200 μl, for example 10 μl, 50 μl, or 100 μl.

According to one embodiment, a distance d between the first container and the second container is comprised between 1 mm and 20 mm, for example 4.5 mm, 9 mm, or 18 mm.

According to one embodiment, the first one-way valve is one of a ball check valve, a swing check valve, a disc check valve, a Tesla valve, a membrane.

According to one embodiment, the second one-way valve is one of a ball check valve, a swing check valve, a disc check valve, a Tesla valve, a membrane.

According to one embodiment, an opening pressure of the first valve, respectively the second valve, is determined based on a suction pressure, respectively a discharge pressure.

According to one embodiment, the actuator comprises a pump.

According to one embodiment, the pump comprises a mechanical piston.

According to one embodiment, the pump acts on an incompressible liquid fluid or on a compressible gaseous fluid to create the suction pressure and the discharge pressure.

According to one embodiment, a filter is positioned at an interface between the actuator and the storage volume, to prevent the inclusion of foreign particles in the volume of transferred fluid.

According to one embodiment, the intermediate storage capacity of the device is configured by means of a volume limiter, for example a piston, a float, or a flexible membrane, closing the inlet of the compressible gaseous fluid to limit a volume of the fluid sucked by the device when the actuator is operated by means of a pump acting on a compressible gaseous fluid.

According to one embodiment, the device further comprises a bridge configured to establish a bidirectional fluid communication between the first container and the second container, the bridge having a hydraulic resistance greater than a hydraulic resistance of the assembly comprising via the inlet channel, the first valve, the storage capacity, the second valve, the outlet channel.

According to one embodiment, the device comprises a coupling interface for the storage capacity of the device, the coupling interface being an opening in the storage capacity which is configured to ensure a sealed fluid coupling with an end piece d further in fluid communication via a channel of the microfluidic chip of the actuator fitted to the opening of the storage capacity, so that the fluid coupling between the end piece and the opening is sealed when the end piece of the actuator is coupled to the opening of the storage capacity.

According to these arrangements, the suction pressure and the discharge pressure created by the actuator are transmitted entirely to the storage capacity via the sealed fluid coupling.

The fluid coupling or a fluid link is said to be sealed when the coupling at the coupling interface enables fluid communication between the two coupled entities without any part of the fluid being able to escape outside the coupling interface, so that all the fluid flowing from one coupled entity to the other reaches the other coupled entity.

According to one embodiment, the opening of the storage capacity comprises an O-ring that ensures sealed fluid coupling between the end piece of the actuator and the opening of the storage capacity when the end piece of the actuator enters the opening of the storage capacity.

According to one embodiment, the opening of the storage capacity is shaped as a cone open toward the outside of the storage capacity, the open cone being configured to receive a corresponding closed cone positioned at the outlet of the end piece of the actuator, so that when the end piece of the actuator enters the opening of the storage capacity, a contact between an outer surface of the closed cone of the end piece and an inner surface of the open cone of the opening ensures sealed fluid coupling between the end piece of the actuator and the opening of the storage capacity.

According to one embodiment, the opening of the storage capacity B may be in the shape of a closed cone, and the end piece of the actuator A may be in the shape of an open cone corresponding to the closed cone of the opening of the storage capacity B, so that when the closed cone of the opening of the storage capacity B enters the inside of the open cone of the end piece of the actuator A, the fluid coupling between the end piece of the actuator A and the opening of the storage capacity B is sealed.

According to one embodiment, a link between the inlet channel and the first container is sealed.

According to one embodiment, a link between the outlet channel and the second container is sealed.

According to these arrangements, the sealing of the link between the inlet channel and the first container and the sealing of the link between the outlet channel and the second container allow for a fully pressure-controlled system, which no longer depends on its orientation relative to the ground or atmospheric pressure. This sealing makes it possible, for example, to consider a use without the presence of gravity (as on an orbital station) or without conventional atmospheric pressure (as in a submarine, an airplane, or a module on the Moon or Mars).

According to another aspect, the invention also concerns an assembly comprising a first container and a second container and a device according to any of the embodiments described above, the inlet channel of the device being placed in fluid communication with the first container and the outlet channel being placed in fluid communication with the second container so as to create a unidirectional fluid connection from the first container to the second container.

According to one embodiment, the assembly according to the invention comprises one or several of the following features, alone or in combination.

According to one embodiment, the first container of the assembly is a reservoir of a microfluidic chip, and the second container of the assembly is another reservoir of the microfluidic chip.

According to one embodiment, the reservoir and the other reservoir of the microfluidic chip are further in fluid communication via a channel of the microfluidic chip.

According to these arrangements, a single-chip closed loop is formed, so that the fluid circulates in a circular manner within the microfluidic chip when an actuator is connected to the device.

According to these arrangements, an intermittent shear stress whose direction is fixed is generated by the flow speed controlled by the actuator, without the need for an external recirculation device through a pump. This shear stress allows reproducing the natural stress experienced by cells and allows them to organize and stretch longitudinally as in the vascular system, thus approaching their in vivo operating mode.

According to one implementation, the microfluidic chip further comprises a central container containing an organ to be perfused by the vascular system thus reproduced, the central container being provided with a nanoporous membrane and the channel of the microfluidic chip passing through this central container.

According to one embodiment, the channel of the microfluidic chip has a hydraulic resistance greater than the hydraulic resistance of the assembly comprising via the inlet channel, the first valve, the storage capacity, the second valve, the outlet channel.

According to one embodiment, the first container is one of the reservoirs of a microfluidic chip and the second container is one of the reservoirs of another microfluidic chip.

According to one embodiment, the other reservoir among the reservoirs of the microfluidic chip and the other reservoir among the reservoirs of the other microfluidic chip are in fluid communication via a fluid bridge, said fluid bridge being a passive fluid bridge, or a device according to any of the embodiments described above.

According to these arrangements, when the fluid bridge is passive, the fluid level is identical in all reservoirs after a given time following implementation of the actuator to transfer a given volume of fluid from the first container to the second container.

According to one implementation, the first reservoir and/or the second reservoir belong to microfluidic chips in a set of microfluidic chips arranged in a standard format, such as the format defined by the ANSI SLAS 4-2004 (R2012) standard, also recognized as ANSI/SBS 4-2004.

According to one embodiment, a link between the inlet channel and the first container is sealed.

According to one embodiment, a link between the outlet channel and the second container is sealed.

According to another aspect, the invention also concerns a network of microfluidic chips interconnected in series comprising a plurality of N microfluidic chips, N being an integer greater than or equal to 2, interconnected by another plurality of N fluid bridges, wherein the plurality of N fluid bridges comprises P passive fluid bridges, and Q devices according to any of the embodiments described above, where P and Q are integers greater than or equal to 1, such that P+Q=N or P+Q=N+1, wherein:
each microfluidic chip of the plurality of N microfluidic chips is connected with at least one other microfluidic chip of the plurality of N microfluidic chips, via a passive fluid bridge, or via a device according to any of the embodiments described above so as to form an assembly according to any of the embodiments described above,
the network comprises at least one assembly according to any of the embodiments described above;
if the network comprises an assembly according to any of the embodiments described above and another assembly according to any of the embodiments described above, one direction of a fluid connection of the assembly of the network and another direction of another fluid connection of the other assembly of the network are identical.

According to one embodiment, a microfluidic chip of the network is connected, via a passive fluid bridge or via a device according to any of the embodiments described above, to an inlet container.

According to one embodiment, a microfluidic chip of the network is connected, via a passive fluid bridge or via a device according to any of the embodiments described above, to an outlet container.

According to one embodiment, the plurality of microfluidic chips are arranged in a standard format, such as the format defined by the ANSI SLAS 4-2004 (R2012) standard, also recognized as ANSI/SBS 4-2004.

According to one embodiment, the fluid bridges of the plurality of N fluid bridges, comprising a subset of devices according to any of the embodiments described above, are arranged in a predetermined configuration which is configured to correspond to a corresponding predetermined arrangement of microfluidic chips, arranged for example in the format defined by the ANSI SLAS 4-2004 (R2012) standard, also recognized as ANSI/SPB 4-2004.

According to one embodiment, the fluid bridges of the plurality of fluid bridges arranged in the predetermined configuration, are preassembled on an assembly frame to which they are securely fastened.

According to another aspect, the invention also concerns a method for creating a unidirectional active fluid connection from a first container to a second container, with at least one device according to any of the embodiments described above, the method comprising the following steps:
placing the inlet channel of the at least one device in the first container;
placing the outlet channel of the at least one device in the second container.

According to another aspect, the invention also concerns a method for transferring a given volume of a fluid with at least one device according to any of the embodiments described above, the method comprising the following steps:
fluidically coupling to the storage capacity of the at least one device, an actuator configured to implement at least one suction and discharge cycle, the suction and discharge cycle comprising a generation of a suction vacuum towards the storage capacity of the at least one device to suck the given volume of fluid from a first container to the intermediate storage capacity, followed by a generation of a discharge pressure in the storage capacity of the at least one device to discharge the given volume of fluid from the intermediate storage capacity to a second container;
implementing at least one suction and discharge cycle of the actuator.

According to one implementation, the cycle is implemented according to a symmetrical period.

According to one implementation, the cycle is implemented according to a period comprising a time pause during which the pressure is kept constant.

According to one implementation, the cycle is implemented according to an asymmetric period.

According to one implementation, the cycle is implemented aperiodically. According to one implementation, the at least one device comprises at least two devices, and the actuator is fluidically coupled in parallel to the storage capacity of the at least two devices, so as to create the suction vacuum and then the discharge pressure, in parallel in the storage capacity of each device of the at least two devices.

According to another aspect, the invention also concerns a method for storing a given amount of fluid in the storage capacity of a device according to any of the embodiments described above, for example before or after implementation of a method according to any of the implementations described above, and for recovering the given amount of fluid, in a manner not synchronized with the storage, the method comprising the following steps:
fluidically coupling to the storage capacity of the at least one device, an actuator configured to implement at least one suction step, then a discharge step, the suction step and the discharge step not being synchronized,
implementing the step of sucking the given volume into the storage capacity of the device,
implementing, in a manner not synchronized with the previous step, the step of discharging the given volume of fluid present in the storage capacity of the device.

For a better understanding, an embodiment and/or implementation of the invention is described with reference to the attached drawings representing, as non-limiting example, an embodiment or implementation respectively of a device and/or a method according to the invention. The same references in the drawings designate similar elements or elements whose functions are similar.

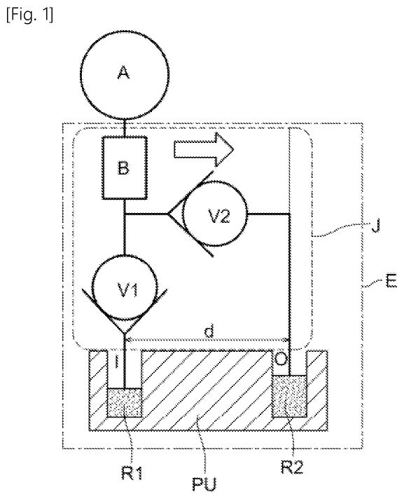

FIG. 1 is a schematic presentation of a fluid connection device according to one embodiment of the invention

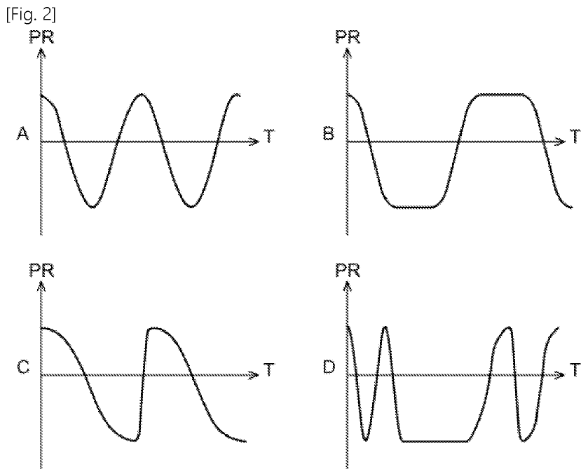

FIG. 2 is a graphical representation of different functions (A, B, C, D) for driving the pressure as a function of time generated by an actuator coupled to the fluid connection device according to the invention

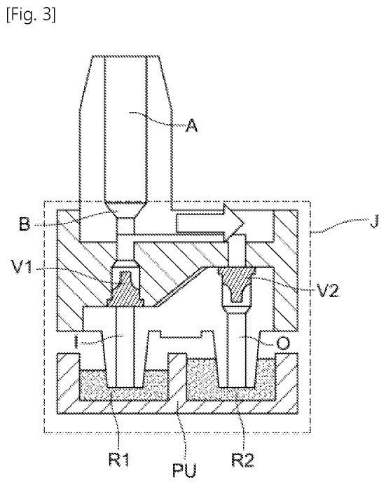

FIG. 3 is another view of a fluid connection device according to one embodiment of the invention

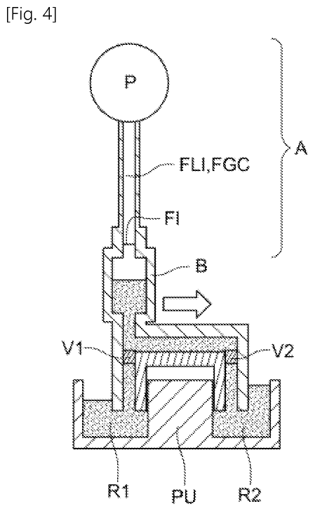

FIG. 4 is a schematic presentation of an embodiment of an actuator of a fluid connection device according to one embodiment of the invention

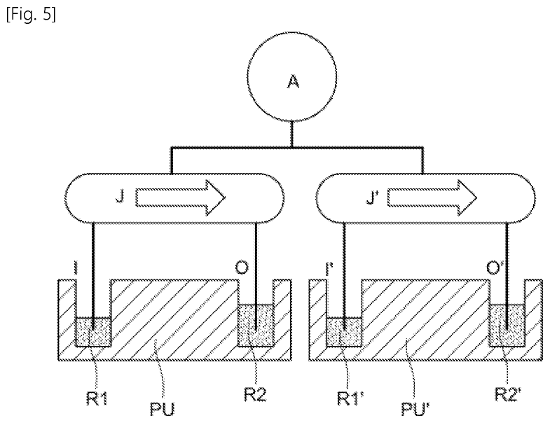

FIG. 5 is a schematic presentation of an implementation of two fluid connection devices according to one embodiment of the invention

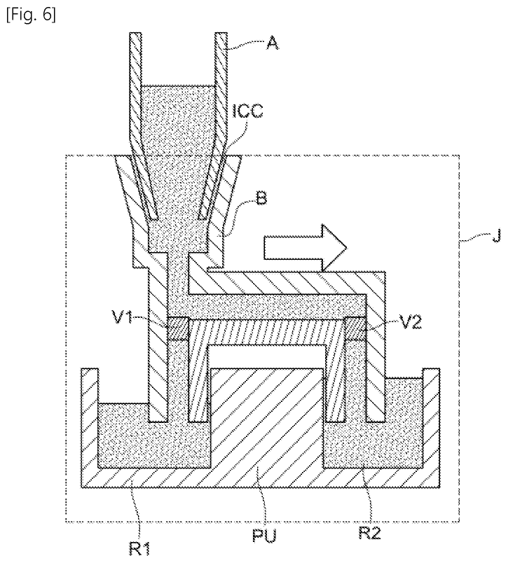

FIG. 6 is a schematic presentation of a coupling between an actuator and a fluid connection device according to one embodiment of the invention

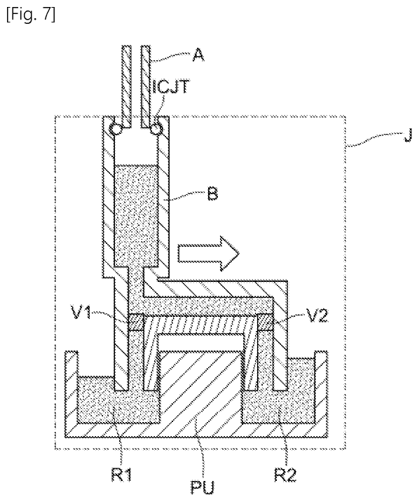

FIG. 7 is a schematic presentation of another coupling between an actuator and a fluid connection device according to one embodiment of the invention

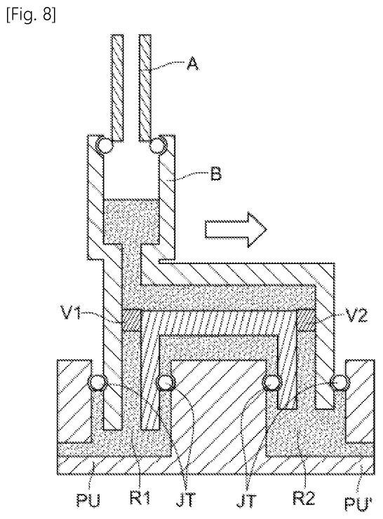

FIG. 8 is a schematic presentation of a coupling between a fluid connection device and the microfluidic containers, according to one embodiment of the invention

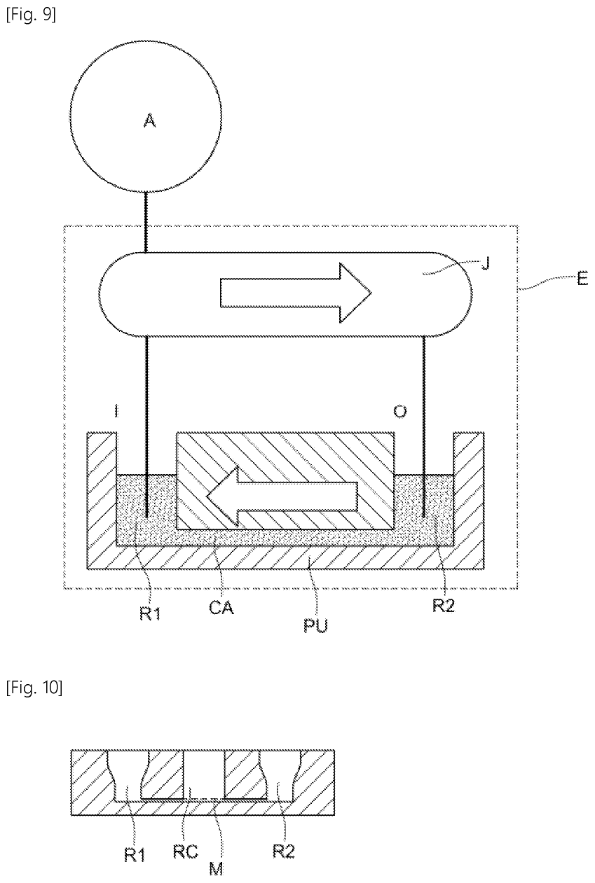

FIG. 9 is a schematic presentation of an implementation of a fluid connection device for implementing a single-chip microfluidic closed loop.

FIG. 10 is a schematic presentation of another implementation of a fluid connection device for implementing a single-chip microfluidic closed loop.

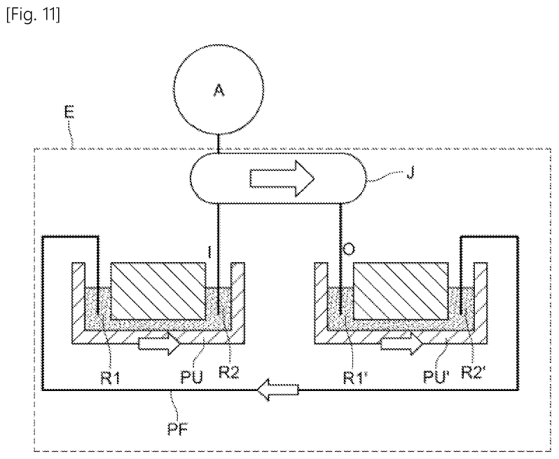

FIG. 11 is a schematic presentation of another implementation of a fluid connection device for implementing a closed loop with two microfluidic chips connected via the fluid connection device, with passive feedback.

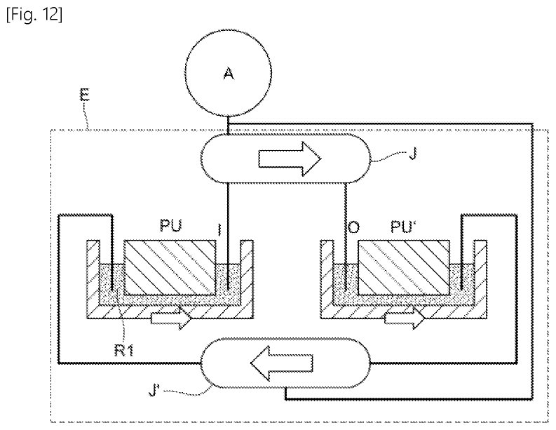

FIG. 12 is a schematic presentation of another implementation of a fluid connection device for implementing a closed loop with two microfluidic chips connected via the fluid connection device, with active feedback using a second fluid connection device.

FIG. 13 is a schematic presentation of another implementation of more than two fluid connection devices for implementing an open loop with two microfluidic chips connected to each other via the fluid connection devices and connected respectively to an inlet container and an outlet container.

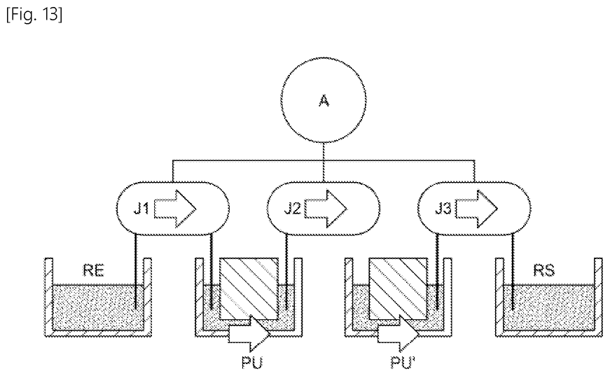

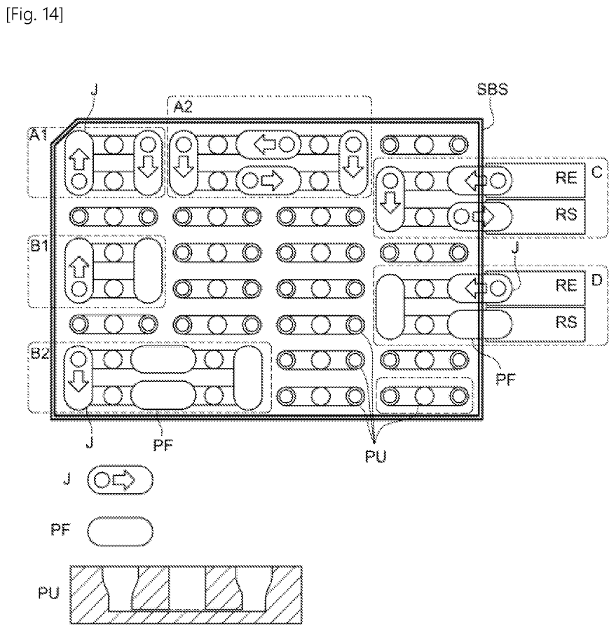

FIG. 14 is a schematic presentation of another implementation of more than two fluid connection devices for implementing different connections between microfluidic chips on a standard support in the format defined by the ANSI SLAS 4-2004 (R2012) standard, also recognized as ANSI/SBS 4-2004.

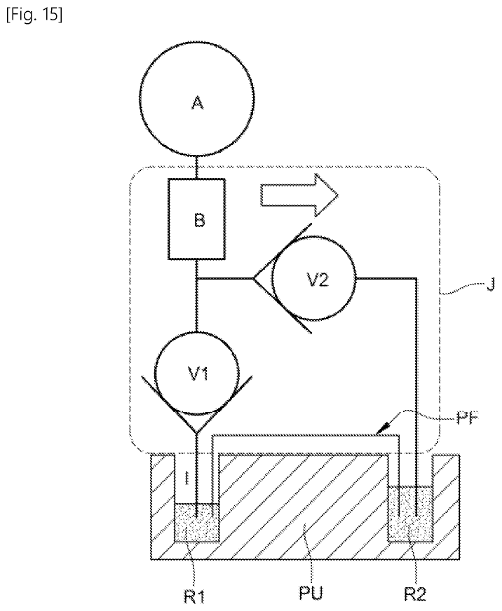

FIG. 15 is a schematic presentation of another implementation of a fluid connection device with an error compensation fluid bridge.

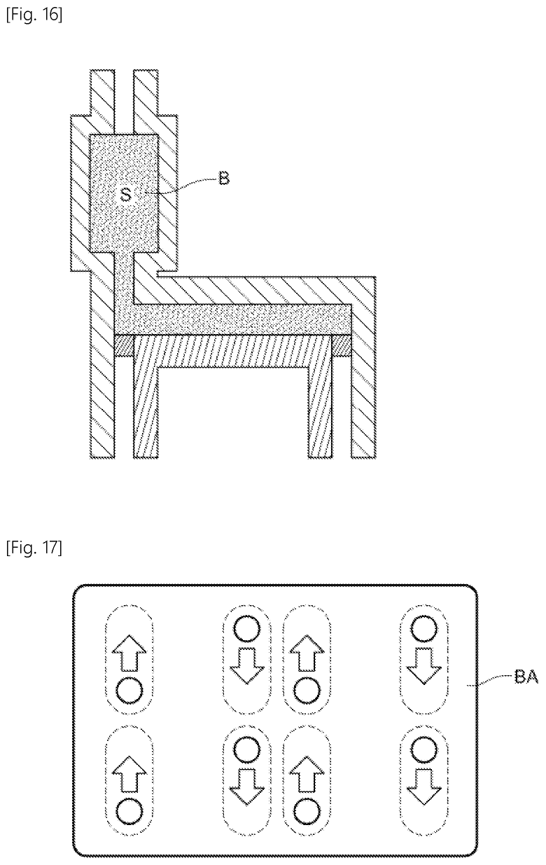

FIG. 16 is a schematic presentation of another implementation of a fluid connection device for storing a given volume of fluid before or after implementation of a fluid connection device.

FIG. 17 is a schematic presentation of a predetermined configuration of devices according to the invention.

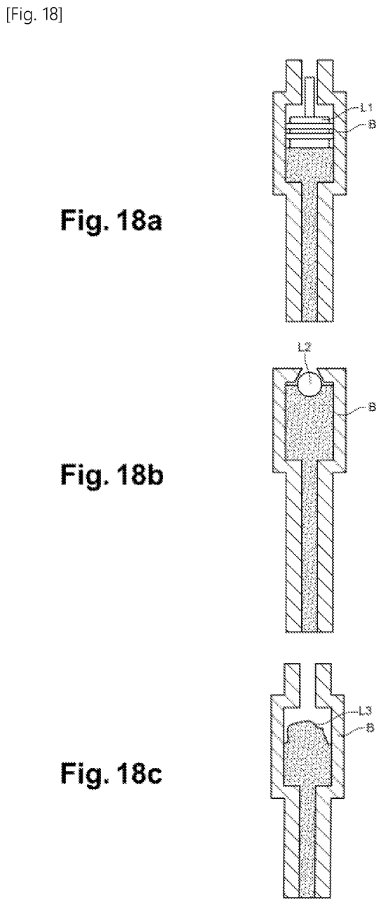

FIG. 18 is a schematic presentation of an intermediate storage capacity of the device, according to three particular embodiments of the invention represented respectively in FIG. 18a, FIG. 18b, and FIG. 18c, in which the intermediate storage capacity is configured to limit a volume of transferred fluid in a predetermined manner.

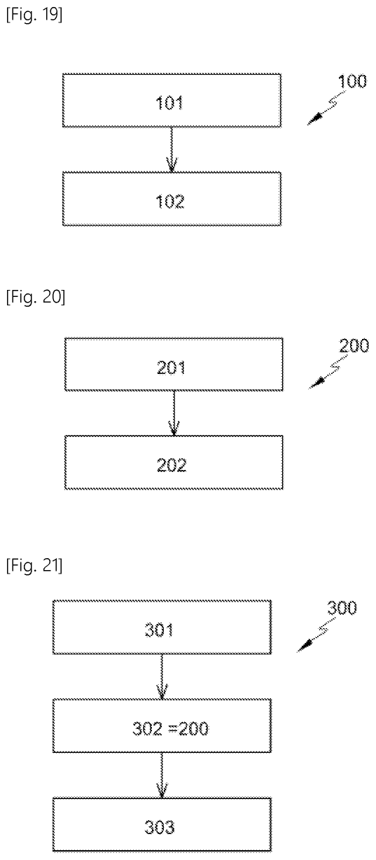

FIG. 19 is a schematic diagram of the sequence of steps of a method for fluidically connecting two containers according to one implementation of a fluid connection device.

FIG. 20 is a schematic diagram of the sequence of steps of a method for transferring a given volume of fluid according to one implementation of at least one fluid connection device.

FIG. 21 is a schematic diagram of the sequence of steps of a method for storing a given volume of fluid before or after implementation of a fluid connection device.

The device according to the invention is intended to enable the interconnection of elementary microfluidic chips to form more complex systems, without adding tubes and dead volume.

To this end, the invention concerns a unidirectional fluid connection elementary device J, which is configured to establish a fluid connection between a first container R1 and a second container R2. FIG. 1 illustrates an embodiment of an elementary device J according to the invention. FIG. 3 is another representation of an elementary device J according to the invention.

With reference to FIGS. 1 and 3, said fluid connection device J is described below. Device J comprises an intermediate fluid storage capacity B, intended to temporarily store a given volume of a fluid taken from the first container R1, while waiting for said given volume of the fluid to be transferred to the second container R2. Device J comprises a fluid inlet channel I, intended to allow said given volume of fluid to be taken from the first container R1. Device J also comprises an outlet channel O for said given volume of fluid, intended to allow said given volume of fluid to be discharged to the second container R2. Device J further comprises a first one-way valve V1 which is configured to provide one-way fluid communication from the inlet channel I to the storage capacity B, and a second one-way valve V2 which is configured to provide one-way fluid communication from the storage capacity B to the outlet channel O.

According to these arrangements, the device J is configured to transfer the given volume of the fluid from the first container R1 to the second container R2, when the inlet channel is placed in fluid communication with the first container R1 and when the outlet channel O is placed in fluid communication with the second container R2.

To perform transfer of the given volume of the fluid from the first container R1 to the second container R2, the device J is actuated by an actuator A fluidically coupled to the intermediate fluid storage capacity B. Actuator A is configured to implement at least one suction and discharge cycle, the suction and discharge cycle comprising a generation of a suction vacuum towards the storage capacity B of the device J to suck the given volume of fluid from the first container R1 to the intermediate storage capacity B, followed by a generation of a discharge pressure in the storage capacity B of the device J to discharge the given volume of fluid from the intermediate storage capacity B to the second container R2.

During the suction phase, the sucked fluid can only come from the inlet channel I due to the one-way nature of the valve V2, whose non-return mechanism prevents the sucked fluid from coming from the outlet channel O.

During the discharge phase, the discharged fluid can only be discharged towards the outlet channel O due to the one-way nature of the valve V1, whose non-return mechanism prevents the fluid from being discharged towards the inlet channel I.

Thus, after a complete cycle of suction and then discharge, the device J, which is activated by the actuator A, has transferred a given volume of fluid from the inlet channel I to the outlet channel O, over a distance d, where d is the distance separating the inlet channel I from the outlet channel O.

According to an exemplary embodiment, the first container R1 is a first microfluidic reservoir, and the second container R2 is a second microfluidic reservoir, the first microfluidic reservoir and the second microfluidic reservoir forming a microfluidic chip PU. A reservoir is said to be microfluidic if it belongs to a microfluidic chip PU or if it has dimensions typical of those of a reservoir of a microfluidic chip. A microfluidic chip consists of at least two reservoirs connected by at least one channel.

For example, the given volume of fluid transferred using the device according to the invention is representative of the microfluidic volumes moved from one microfluidic reservoir to another microfluidic reservoir, belonging to the same microfluidic chip, or to two separate microfluidic chips, in the range of a few μl to a few hundred μl, typically comprised between 5 μl and 200 μl, for example 10 μl, 50 μl, 100 μl, or 150 μl.

More particularly, a distance d between the first container and the second container is comprised between 1 mm and 20 mm, for example 4.5 mm, 9 mm, or 18 mm. These orders of magnitude correspond in particular to the standard formats of a set of microfluidic chips, as defined by the ANSI SLAS 4-2004 (R2012) standard, also recognized as ANSI/SBS 4-2004.

The first one-way valve V1 and/or the second one-way valve V2 may take the form, for example, of a ball check valve, a swing check valve, a disc check valve, a Tesla valve, or a simple membrane. The non-return mechanism of the first valve V1 may be different from the non-return mechanism of the second valve V2. An opening pressure of the first valve V1, respectively the second valve V2, is determined based on a suction pressure, respectively a discharge pressure.

For example, the actuator A, illustrated in FIG. 4, may comprise a pump P, configured, for example, to act on an incompressible liquid fluid FLI, or on a compressible gaseous fluid FGC, to successively create the suction pressure and then the discharge pressure.

Pump P may comprise a mechanical piston.

When the pump P is configured to act on a compressible gas, the volume of fluid transferred upon activation of the device J will depend on the opening pressures and the compressibility of the gas used by the pump P. Nevertheless, the use of a compressible gas enables remote activation of the device J without exchanging liquid with the outside of the device J, which simplifies cleaning and prevents liquid contaminations.

According to a particular exemplary embodiment represented in FIG. 18, the intermediate storage capacity B of the device J is configured by means of a volume limiter L1, L2, L3, closing the inlet of the compressible gas to limit a volume of the fluid sucked by the device J when the actuator A is operated by means of a pump P acting on a compressible gas. Said volume limiter may, for example, be a piston L1, as represented in FIG. 18a, a float L2 as represented in FIG. 18b, or a flexible membrane L3 as represented in FIG. 18c.

When the pump P is configured, for example, to act on an incompressible liquid, the volume of fluid transferred upon activation of the device J is fixed and independent of the suction and discharge pressures, as long as these are greater than the opening pressure of the valves V1, V2; however, this requires local actuation, such as for example an integrated mechanical piston, to prevent external contamination and manage leakage during disconnection.

According to an exemplary embodiment, a filter FI is positioned at an interface between the actuator A and the storage volume B, to prevent the inclusion of foreign particles in the volume of transferred fluid.

Several devices J, J′ according to the invention can be activated in parallel by a single actuator A, as illustrated in FIG. 5. According to this arrangement, the suction and then discharge pressure generated by the actuator A is exerted in parallel on the storage capacity of each device J, J′, so that each device J, J′ simultaneously transfers a given volume of fluid respectively from a first container R1 to a second container R2, belonging, for example, to a first microfluidic chip PU, and from another first container R1′ to another second container R2′, belonging, for example, to another first microfluidic chip PU′.

The actuator A that drives the device(s) J, J′ can sequence the suction and discharge periods in several ways, leading to several effects. The actuation system can be driven by any pressure or displacement function over time. For example, FIG. 2 shows four different profiles A, B, C, D of the pressure evolution PR, represented on the ordinate, as a function of time T represented on the abscissa during the actuation cycle sequence of the device(s) J, J′:
A—symmetrical periodic driving. This actuation generates an equivalent, symmetrical pressure variation at the inlet I and the outlet O. This is useful if the aim is to generate an equivalent flow at several locations in the system.
B—periodic driving with pauses. This actuation is similar to A, but pauses in the system allow the different regions of the fluid circuit to regain static pressure equilibrium before being subjected to the next fluid transfer.
C—Asymmetric periodic driving. This actuation allows differentiating between the inlet and outlet flows, which makes it possible to modulate the effects of the flows on microfluidic cells, for example.
D—Multi-periodic or aperiodic driving. This actuation allows simulating more complex or erratic events, such as cardiac arrest or fibrillation, for example.

As illustrated in FIGS. 6 and 7, the intermediate storage capacity B of the device J comprises a coupling interface ICC, ICJT, which is configured to enable a sealed fluid coupling between the actuator A and the storage capacity B, so that the suction pressure and discharge pressure generated by the actuator A are fully transmitted to the storage capacity B via said sealed fluid coupling. According to a particular embodiment, the coupling interface ICC, ICJT is an opening of the storage capacity B configured to receive an end piece of the actuator A, which is fitted to the opening of the storage capacity B so as to ensure a sealed fluid coupling when the end piece of the actuator A enters the opening of the storage capacity B.

The fluid coupling or a fluid link is said to be sealed when the coupling at the coupling interface enables fluid communication between the two coupled entities without any part of the fluid being able to escape outside the coupling interface, so that all the fluid flowing from one coupled entity to the other reaches the other coupled entity.

According to a particular embodiment of the coupling interface ICC schematically represented in FIG. 6, the opening of the storage capacity B is shaped as a cone open toward the outside of the storage capacity B, the open cone being configured to receive a corresponding closed cone positioned at the outlet of the end piece of the actuator A, so that when the end piece of the actuator A enters the opening of the storage capacity B, a contact between an outer surface of the closed cone of the end piece and an inner surface of the open cone of the opening ensures the sealed coupling between the end piece of the actuator A and the opening of the storage capacity B. According to another embodiment, the roles of the opening of the storage capacity B and the end piece of the actuator A can be reversed, i.e. the opening of the storage capacity B may be in the shape of a closed cone, and the end piece of the actuator A may be in the shape of an open cone corresponding to the closed cone of the opening of the storage capacity, so that when the closed cone of the opening of the storage capacity enters the inside of the open cone of the end piece of the actuator A, the fluid coupling between the end piece of the actuator A and the opening of the storage capacity B is sealed.

According to another particular embodiment of the coupling interface ICJT represented schematically in FIG. 7, the opening of the storage capacity B comprises an O-ring that ensures the sealed fluid coupling between the end piece of the actuator A and the opening of the storage capacity B when the end piece of the actuator A enters the opening of the storage capacity B.

The fluid link of the device J with the first container R1 is achieved via the inlet channel I. According to a particular exemplary embodiment, the link between the first container R1 and the inlet channel I may be sealed, for example, using an O-ring fitted to the size of the inlet channel I as illustrated in FIG. 8, or via a cone-cone interface not represented in FIG. 8. However, it may also be free, i.e., not sealed, so that the fluid pressure in the first container is ambient pressure; the fluid link is then simpler to achieve.

Similarly, the fluid link between the device J and the second container R2 is achieved via the outlet channel O. According to a particular exemplary embodiment, the link between the second container R2 and the outlet channel O may be sealed, for example, using an O-ring fitted to the size of the outlet channel O as illustrated in FIG. 8, or via a cone-cone interface not represented in FIG. 8. However, it can also be free, i.e. not sealed, so that the fluid pressure in the second container is ambient pressure; the fluid link is then simpler to achieve.

In FIG. 8, the fluid link between the device J and each of the containers R1, R2 is sealed. However, according to another exemplary embodiment not represented in FIG. 8, a fluid link with one of the containers R1, R2 can be sealed and the other fluid link with the other container can be free. Finally, according to another exemplary embodiment also not represented in FIG. 8, the fluid link between the device J and each of the containers R1, R2 can be free.

The sealing of the link between the inlet channel I and the first container R1, and the sealing of the link between the outlet channel O and the second container R2, allow for a fully pressure-controlled system, which no longer depends on its orientation relative to the ground or atmospheric pressure. This sealing makes it possible, for example, to consider a use without the presence of gravity (as on an orbital station) or without conventional atmospheric pressure (as in a submarine, an airplane, or a module on the Moon or Mars).

According to one aspect, an exemplary embodiment of which is illustrated in FIG. 1, the invention concerns a set E comprising a first container R1 and a second container R2 and a device J according to any of the embodiments described above, the inlet channel 1 of the device J being placed in fluid communication with the first container R1 and the outlet channel O being placed in fluid communication with the second container R2 so as to create a unidirectional fluid connection from the first container R1 to the second container R2.

According to an exemplary embodiment, the first container R1 of the set is a reservoir of a microfluidic chip PU, and the second container R2 is another reservoir of the microfluidic chip PU.

In particular, as illustrated in FIG. 9, the reservoir and the other reservoir of the microfluidic chip PU may also be in fluid communication via a channel CA of the microfluidic chip PU. Thus, a single-chip closed loop is formed, so that the fluid circulates in a circular manner within the microfluidic chip PU when an actuator is connected to the device.

This circular circulation has the advantage of subjecting cells—for example, endothelial cells—to intermittent shear stress in a fixed direction, without the need for an external recirculation system involving a pump. This shear stress—generated by the flow speed, which can be controlled by the actuator A—reproduces the natural stress experienced by these cells and allows them to organize and stretch longitudinally as in the vascular system, thus approaching their in vivo functionality, which is dependent on this morphology. It should be noted that in this case, as illustrated in FIG. 10, the device J could have to span a central reservoir RC equipped with a nanoporous membrane M, containing an organ to be perfused by the vascular system thus reproduced, said central reservoir being located between the first and second reservoirs R1, R2 of the microfluidic chip PU.

The device can also be used to connect several microfluidic chips together in a closed loop, thus constituting a multi-organ system connected by a single vascular system that shares and recirculates nutrients, toxins, pharmaceutical compounds, etc. The loop can be closed by completing the system with a passive return, such as a simple fluid bridge intended to restore, through communicating vessels, a liquid level common to all the chips. Thus, according to an exemplary embodiment of a set E illustrated in FIG. 11, the first container, into which the inlet interface I of the device J, is one of the two reservoirs R1, R2 of a microfluidic chip PU and the second container, into which the outlet interface O of the device J enters, is one of the two reservoirs R1′, R2′ of another microfluidic chip PU′; the other reservoir R1 among the two reservoirs R1, R2 of the microfluidic chip PU, and the other reservoir R2′ among the two reservoirs R1′, R2′ of the other microfluidic chip PU′ are in fluid communication via a fluid bridge PF. Said fluid bridge PF may be a passive fluid bridge, according to the embodiment represented in FIG. 11. Said fluid bridge PF may also be another device J′, according to any of the embodiments described above, according to another embodiment represented in FIG. 12.

According to these arrangements, when the fluid bridge PF is passive, the fluid level is identical in all the reservoirs after a given time following implementation of the actuator A to transfer a given volume of the fluid from the first container to the second container. This embodiment has the advantage of being simple, but it does not impose the same flow conditions on the microfluidic chip PU as on the other microfluidic chip PU′, because the passive fluid bridge PF restores the levels at a different speed than the device J imposed for transferring the given volume of the fluid from the microfluidic chip PU to the other microfluidic chip PU′.

When the fluid bridge PF is active, i.e. produced with another device J′ according to the invention, as represented in FIG. 12, the device J transfers the given volume of fluid from the microfluidic chip PU to the other microfluidic chip PU′, and simultaneously, the other device J′ transfers an identical given volume from the other microfluidic chip PU′ to the microfluidic chip PU. This double transfer can be carried out according to periodic cycles, driven by a rate of the actuator A which is applied simultaneously to both devices J and J′. This system has the advantage of imposing identical flow conditions on all the microfluidic chips, which makes it possible, for example, to create uniform shear stress conditions throughout the vascular system thus formed. However, this embodiment introduces a weakness in its sensitivity to the accuracy of the given volumes of transferred fluid. Indeed, if the volumes of fluid transferred by the two devices differ slightly—this which is likely—one microfluidic chip will eventually dry out and the other microfluidic chip will overflow after a finite number of cycles. This phenomenon is amplified by the fact that several devices J are in parallel on the same actuator A and that the deviations, or errors, in volumes transferred by each device are accumulated.

This defect can be corrected by an embodiment of the device J comprising an error compensation means described below. According to this embodiment, illustrated as an example in FIG. 15, the device J further comprises a fluid bridge PF configured to establish a bidirectional fluid communication between the first container R1 and the second container R2, the bridge having a hydraulic resistance greater than a hydraulic resistance of the set comprising, via the inlet channel I, the first valve V1, the storage capacity B, the second valve V2, the outlet channel O. Thus, the fluid bridge PF contributes to bringing passively, the microfluidic chip(s) toward a common fluid level by a communicating vessel effect. Due to the fact that the passive fluid bridge PF integrated into each device J has a hydraulic resistance greater than the hydraulic resistance of all the other components of said device J, i.e. the inlet channel I, the first valve V1, the storage capacity B, the second valve V2, the outlet channel O, the fluid flows in the direction imposed by the device(s) J used to form the set of interconnected microfluidic chips. Thanks to the passive fluid bridge(s) PF integrated into each device J interconnecting the set of microfluidic chips PU, PU′, any differential height of the fluid level in the different microfluidic chips of the considered set is leveled over time. An additional solution to the passive fluid bridge PF integrated into the device J according to the invention, to achieve equalization of fluid levels in the various microfluidic chips, consists in introducing pauses in the actuation cycle of the devices to give the fluid levels time to be homogenized.

The devices J according to the invention can thus be used to interconnect microfluidic chips PU, PU′ and containers in various modular configurations, such as configurations A1, A2, B1, B2, C, D, represented in FIG. 14, as an example. According to one embodiment of configurations A1, A2, B1, B2, C, D of a set of microfluidic chips, the microfluidic chips are arranged in a standard format, such as the format defined by the ANSI SLAS 4-2004 (R2012) standard, also recognized as ANSI/SBS 4-2004. The microfluidic chips can be arranged in 24-well SBS format plates with a well spacing of 18 mm, in 384-well SBS format plates with a well spacing of 4.5 mm, in 1536-well SBS format plates with a well spacing of 2.25 mm, or even in 96-well or 6-well SBS format plates.

For example, FIG. 14 represents, on a 96-well SBS format plate:
two multi-chip microfluidic closed loops A1, A2, with active return, based on the use of several devices J to ensure the fluid circulation in one direction and in the return direction;
two multi-chip microfluidic closed loops B1, B2, with passive return based on the use of several devices J to ensure the circulation of the fluid in a single direction, the return being based on passive fluid bridges;
An active open loop C configured to transfer the fluid between an inlet reservoir RE and an outlet reservoir RS, passing through a chain of microfluidic chips interconnected in series by devices J according to the invention;
A passive open loop D configured to transfer the fluid between another inlet reservoir RE and another outlet reservoir RS, passing through another chain of microfluidic chips interconnected in series by devices J according to the invention for a portion of the chain, and for another portion of the chain by simple passive fluid bridges PF;

These different configurations A1, A2, B1, B2, C, D, of microfluidic chip sets thus each constitute a network A1, A2, B1, B2, C, D of microfluidic chips interconnected in series, comprising a plurality of N microfluidic chips, N being an integer greater than or equal to 2, interconnected by another plurality of N fluid bridges, wherein the plurality of N fluid bridges comprises P passive fluid bridges, and Q devices according to any of the embodiments described above, where P and Q are integers greater than or equal to 1, such that P+Q=N, or P+Q=N+1 wherein:
each microfluidic chip of the plurality of N microfluidic chips is connected with at least one other microfluidic chip of the plurality of N microfluidic chips via a passive fluid bridge PF, or via a device J according to the invention so as to form a set E according to the embodiment described above,
the network comprises at least one set E;
if the network comprises a set E and another set E, a direction of a fluid connection of the set E of the network A1, A2, B1, B2, C, D and another direction of another fluid connection of the other set E of the network A1, A2, B1, B2, C, D are identical.

The network A1, A2, B1, B2 is closed if P+Q=N; the network C, D is open if P+Q=N+1.

In particular, a microfluidic chip PU of the open network C, D is connected, via a passive fluid bridge PF or via a device J according to the invention, to an inlet container RE.

More specifically, a microfluidic chip PU of the open network C, D is connected, via a passive fluid bridge or via a device J according to the invention, to an outlet container RS.

FIG. 13 illustrates in particular an example of an open network with two microfluidic chips PU, PU′ and three devices J1, J2, J3 according to any of the embodiments of the invention described above, said devices being activated by a single actuator A, the network interconnecting an inlet container RE, and an outlet container RS.

According to a particular example, the fluid bridges of the plurality of N fluid bridges, comprising a subset of devices according to the invention, are arranged in a predetermined configuration configured to correspond to a corresponding predetermined arrangement of microfluidic chips, arranged for example in the format defined by the ANSI SLAS 4-2004 standard (R2012), also recognized as ANSI/SPB 4-2004. An example of a predetermined configuration is illustrated in FIG. 17.

According to an exemplary embodiment, the fluid bridges of the plurality of fluid bridges arranged according to said predetermined configuration are preassembled on an assembly frame BA to which they are securely fastened, with fixed distances, for a dedicated application.

According to one aspect, the invention also concerns a method 100 for creating a unidirectional active fluid connection from a first container R1, R1′, RE to a second container R2, R2′, RS, with at least one device J, J1, J2, J3, according to one of the embodiments described above, the method comprising the following steps, schematically illustrated in FIG. 19:

placing 101 the inlet channel I of the at least one device J, J1, J2, J3 in the first container R1;

placing 102 the outlet channel O of the at least one device J, J1, J2, J3 in the second container R2.

According to another aspect, the invention also concerns a method 200 for transferring a given volume of fluid with at least one device J, J1, J2, J3 according to any of the embodiments described above, the method comprising the following steps, schematically illustrated in FIG. 20:
fluidically coupling 201 to the storage capacity B of the at least one device J, J1, J2, J3, an actuator A configured to implement at least one suction and discharge cycle, the suction and discharge cycle comprising a generation of a suction vacuum towards the storage capacity B of the at least one device J, J1, J2, J3 to suck the given volume of the fluid from a first container R1, R1′, RE to the intermediate storage capacity B, followed by a generation of a discharge pressure in the storage capacity B of the at least one device J, J1, J2, J3 to discharge the given volume of fluid from the intermediate storage capacity B to a second container R2, R2′, RS;
implementing 202 at least one suction and discharge cycle of the actuator A.

According to one exemplary implementation, the cycle is implemented according to a symmetrical period, typically represented by the graph A in FIG. 2.

According to another exemplary implementation, the cycle is implemented according to a period comprising a time pause during which the pressure is kept constant, typically represented by the graph B in FIG. 2.

According to another exemplary implementation, the cycle is implemented according to an asymmetrical period, represented for example by the graph C in FIG. 2.

According to another exemplary implementation, the cycle is implemented aperiodically, represented for example by the graph D in FIG. 2. According to another aspect, the invention also concerns a method 300 for storing, in the storage capacity of a device J, J1, J2, J3 according to any of the embodiments described above, a given amount of fluid before or after implementation of the method 200 for transferring a given volume of a fluid according to any of the embodiments described above, the method 300 comprising the following steps, schematically illustrated in FIG. 21:
storing 301 the given volume in the storage capacity B of the device J, J1, J2, J3,
implementing 302 the method 200 for transferring the given volume with the device J, J1, J2, J3
recovering 303 the given volume of fluid present in the storage capacity B of the device J, J1, J2, J3.

Thus, the device J according to any of the embodiments described above can be used to store an amount of fluid before or after its use in an open-loop or closed-loop circuit of microfluidic chips. The volume S of the storage capacity B, represented schematically in FIG. 16, is constant, which can be used in different ways; for example, before use:
Preparing a medium renewal of the value of Volumes B in the system. Example: if the volume B is 100 μL and two connected chips represent 100 μL each, the active return circuit contains 400 μL in total. Thus, replacing devices J with new devices with fresh medium amounts to a 50% medium change, or a single 25% change, which are fairly common values.
Aliquoting a compound for introduction into the culture/vascular system medium of a multi-organ system. The pre-clinical pharmaceutical compounds are complex to maintain and often require freezing. Frozen aliquots in a device J would allow for thawing to well-defined values suitable for microfluidic chip assays.
Storing/preparing cells ready for injection into a microfluidic system
Storing lyophilized medium, which can be reconstituted with ultrapure water before use

Or, for example, after use, with recovery of volumes to be titrated/analyzed without disrupting the circuit by replacing the device.

1. A unidirectional fluid connection device, the device comprising an intermediate fluid storage capacity configured to temporarily store a given volume of a fluid, the device comprising an inlet channel for the fluid coming into the device and an outlet channel for the fluid going out of the device, the device further comprising a first one-way valve which is configured to provide a one-way fluid communication from the inlet channel to the storage capacity, the device further comprising a second one-way valve which is configured to provide a one-way fluid communication from the storage capacity to the output channel.

2. The device according to claim 1, comprising a coupling interface for the storage capacity of the device, the coupling interface being an opening of the storage capacity configured to ensure a sealed fluid coupling with an end piece of an actuator fitted to the opening of the storage capacity, so that the fluid coupling between the end piece and the opening is sealed when the end piece of the actuator is coupled to the opening of the storage capacity.

3. A set comprising a first container and a second container and a device according to claim 1, the inlet channel of the device being placed in fluid communication with the first container and the outlet channel being placed in fluid communication with the second container so as to create a unidirectional fluid connection from the first container to the second container.

4. The set according to claim 3, wherein the first container is a reservoir of a microfluidic chip, and the second container is another reservoir of the microfluidic chip.

5. The set according to claim 4, wherein the reservoir and the other reservoir of the microfluidic chip are further in fluid communication via a channel of the microfluidic chip.

6. The set according to claim 5, wherein the channel of the microfluidic chip has a hydraulic resistance greater than a hydraulic resistance of the set comprising, via the inlet channel, the first valve, the storage capacity, the second valve, the outlet channel.

7. The set according to claim 3, wherein the first container is one of the reservoirs of a microfluidic chip and the second container is one of the reservoirs of another microfluidic chip.

8. The set according to claim 7, wherein another reservoir among the reservoirs of the microfluidic chip, and another reservoir among the reservoirs of the other microfluidic chip are in fluid communication via a fluid bridge, the fluid bridge being a passive fluid bridge, or a device comprising an intermediate fluid storage capacity configured to temporarily store a given volume of a fluid, the device comprising an inlet channel for the fluid coming into the device and an outlet channel for the fluid going out of the device, the device further comprising a first one-way valve which is configured to provide a one-way fluid communication from the inlet channel to the storage capacity, the device further comprising a second one-way valve which is configured to provide a one-way fluid communication from the storage capacity to the output channel.

9. The set according to claim 3, wherein a link between the inlet channel and the first container is sealed.

10. The set according to claim 3, wherein a link between the outlet channel and the second container is sealed.

11. A network of microfluidic chips interconnected in series comprising a plurality of N microfluidic chips, N being an integer greater than or equal to 2, interconnected by another plurality of N fluid bridges, wherein the plurality of N fluid bridges comprises P passive fluid bridges, and Q devices comprising an intermediate fluid storage capacity configured to temporarily store a given volume of a fluid, the device comprising an inlet channel for the fluid coming into the device and an outlet channel for the fluid going out of the device, the device further comprising a first one-way valve which is configured to provide a one-way fluid communication from the inlet channel to the storage capacity, the device further comprising a second one-way valve which is configured to provide a one-way fluid communication from the storage capacity to the output channel, wherein P and Q are integers greater than or equal to 1, such that P+Q=N or P+Q=N+1, wherein:
each microfluidic chip of the plurality of N microfluidic chips is connected to at least one other microfluidic chip of the plurality of N microfluidic chips, via a passive fluid bridge or via a device comprising an intermediate fluid storage capacity configured to temporarily store a given volume of a fluid, the device comprising an inlet channel for the fluid coming into the device and an outlet channel for the fluid going out of the device, the device further comprising a first one-way valve which is configured to provide a one-way fluid communication from the inlet channel to the storage capacity, the device further comprising a second one-way valve which is configured to provide a one-way fluid communication from the storage capacity to the output channel so as to form a set according to claim 10,
the network comprises at least one set according to claim 10;
if the network comprises a set according to claim 10 and another set according to claim 10, one direction of a fluid connection of the set of the network and another direction of another fluid connection of the other set of the network are identical.

12. A method for creating a unidirectional active fluid connection from a first container to a second container, with at least one device according to claim 1, the method comprising the following steps:
placing the inlet channel of the at least one device in the first container;
placing the outlet channel of the at least one device in the second container.

13. A method for transferring a given volume of a fluid with at least one device according to claim 1, the method comprising the following steps:
fluidically coupling to the storage capacity of the at least one device, an actuator configured to implement at least one suction and discharge cycle, the suction and discharge cycle comprising a generation of a suction vacuum towards the storage capacity of the at least one device to suck the given volume of fluid from a first container to the intermediate storage capacity, followed by a generation of a discharge pressure in the storage capacity of at least one device for discharging the given volume of fluid from the intermediate storage capacity to a second container;
implementing at least one suction and discharge cycle of the actuator.

14. A method for storing in the storage capacity of a device according to claim 1, a given amount of fluid, and for recovering the given amount of fluid, in a manner not synchronized with the storage, the method comprising the following steps:
fluidically coupling to the storage capacity of the at least one device, an actuator configured to implement at least one suction step, then a discharge step, the suction step and the discharge step not being synchronized,
implementing the step of sucking the given volume in the storage capacity of the device,
implementing, in a manner not synchronized with the previous step, the step of discharging the given volume of fluid present in the storage capacity of the device.
